(19) United States
(12) Patent Application Publication
(10) Pub. No.: US 2022/0044797 A1
(43) Pub. Date: Feb. 10, 2022

(54) MEDICAL INFORMATION PROCESSING APPARATUS, MEDICAL INFORMATION PROCESSING METHOD, AND MEDICAL INFORMATION PROCESSING SYSTEM

(71) Applicant: Canon Medical Systems Corporation, Otawara-shi (JP)

(72) Inventors: Yusuke KANO, Nasushiobara (JP); Anri SATO, Nasushiobara (JP)

(73) Assignee: Canon Medical Systems Corporation, Otawara-shi (JP)

(21) Appl. No.: 17/305,956

(22) Filed: Jul. 19, 2021

(30) Foreign Application Priority Data
Aug. 6, 2020 (JP) 2020-133895

(51) Int. Cl. G16H 40/63; G16H 40/20

(52) U.S. Cl. CPC G16H 40/63; G16H 40/20

(57) ABSTRACT

According to one embodiment, a medical information processing apparatus includes processing circuitry. The processing circuitry acquires monitoring data of a patient. The processing circuitry calculates a personal condition level for a physical condition of the patient using the monitoring data. The processing circuitry calculates a level of need for intervention indicative of a degree of need for a medical intervention for the patient, based on the personal condition level and a group condition level for a patient group including the patient. The processing circuitry determines whether or not the level of need for intervention of the patient satisfies a predetermined condition.

### CROSS-REFERENCE TO RELATED APPLICATIONS

This application is based upon and claims the benefit of priority from Japanese Patent Application No. 2020-133895, filed Aug. 6, 2020, the entire contents of which are incorporated herein by reference.

### FIELD

Embodiments described herein relate generally to a medical information processing apparatus, a medical information processing method, and a medical information processing system.

### BACKGROUND

There has been an ever-increasing need recently for systems intended for remote monitoring, where health conditions of patients distant from a medical facility (e.g., a patient at home) are monitored and medical staff is notified once a specific event is detected. Such systems for example check vital data of a patient to determine a physical condition level and, if this physical condition level is found to meet a predetermined condition, transmit an event notification on the assumption that an event has occurred. Remote monitoring allows for early recognition and treatment of deteriorated physical conditions of patients, and accordingly, an improved prognosis of the patients can be expected.

On the other hand, prioritizing early recognition of deteriorated physical conditions would lead to a situation where symptoms that do not require very urgent care are also subject to medical examination. As such, if many patients are targeted within a particular period, the medical staff will be forced to conduct diagnosis for these patients in a short time and must bear an increased burden.

### BRIEF DESCRIPTION OF THE DRAWINGS

FIG. 1 is a block diagram showing a medical information processing system according to an embodiment.

FIG. 2 is a flowchart showing an intervention determining process of a medical information processing apparatus according to a first embodiment.

FIG. 3 is a table according to a first exemplary setting of levels of need for intervention and a threshold, as used in the first embodiment.

FIG. 4 is a conceptual diagram of a process for specifying a patient in need of intervention, performed by a determination function based on contents of the table in FIG. 3.

FIG. 5 is a table according to the first exemplary setting of the levels of need for intervention and the threshold that reflect calculation in a process subsequent to the situation indicated by the table in FIG. 3.

FIG. 6 is a conceptual diagram of a process for specifying a patient in need of intervention, performed by the determination function based on contents of the table in FIG. 5.

FIG. 7 is a conceptual diagram of a second exemplary setting of levels of need for intervention and a threshold, as used in the first embodiment.

FIG. 8 is a conceptual diagram of a third exemplary setting of levels of need for intervention and a threshold, as used in the first embodiment.

FIG. 9 is a diagram illustrating one example of notification to a user terminal, performed by a notification function.

FIG. 10 is a block diagram showing a medical information processing apparatus according to a second embodiment.

### DETAILED DESCRIPTION

In general, according to one embodiment, a medical information processing apparatus includes processing circuitry. The processing circuitry acquires monitoring data of a patient. The processing circuitry calculates a personal condition level for a physical condition of the patient using the monitoring data. The processing circuitry calculates a level of need for intervention indicative of a degree of need for a medical intervention for the patient, based on the personal condition level and a group condition level for a patient group including the patient. The processing circuitry determines whether or not the level of need for intervention of the patient satisfies a predetermined condition.

A medical information processing apparatus, a medical information processing method, and a medical information processing system according to embodiments will be described with reference to the drawings. The description of the embodiments will assume that components or portions having the same reference signs operate in the same manner, and redundant explanations will be omitted as appropriate.

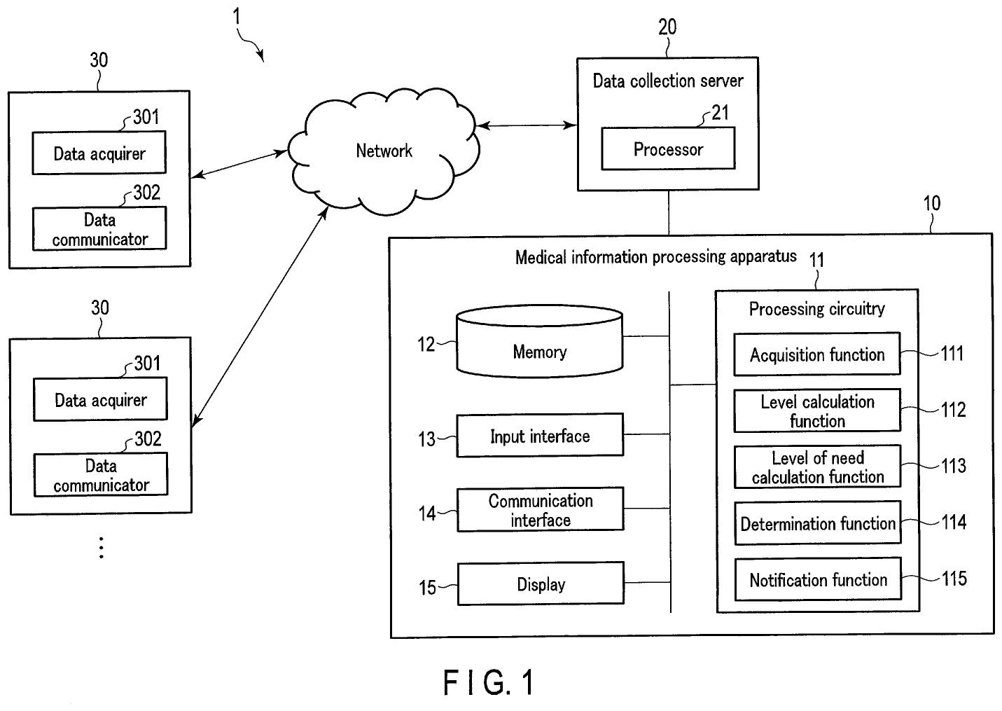

FIG. 1 will be referred to for the medical information processing system according to one embodiment.

The medical information processing system according to the embodiment, denoted by reference sign “1”, includes a medical information processing apparatus 10, a data collection server 20, and one or more user terminals 30. While FIG. 1 shows two user terminals 30, the number of the user terminals 30 is not limited.

Each user terminal 30 includes a data acquirer 301 and a data communicator 302.

The data acquirer 301 acquires monitoring data of a respective patient. For example, assuming that the patient carries in his/her body an implanted device such as a pacemaker, an implantable cardioverter defibrillator (ICD), or an insertable cardiac monitor (ICM), the monitoring data here may be data associable with the patient's health condition and be wirelessly transmitted from the pacemaker, the ICD, or the ICM. Such monitoring data includes a heart rate, arrhythmia information, electrocardiogram information, device information, and so on. The monitoring data may also contain data indicative of a weight, a body fat percentage, a blood pressure, an activity amount, GPS-based location information, etc. As appropriate, values of the weight, the body fat percentage, the blood pressure, and the like may be measured by the patient himself/herself using home measurement instruments such as a weight scale and a sphygmomanometer, and the user terminal 30 may acquire these values through input by the patient. For the activity amount and the location information, values that have been measured by, for example, one or more wearable devices may be used. The monitoring data may further contain data indicative of a symptom (chief complaint) that has been input by the patient himself/herself to a smartphone, a tablet PC, or the like.

The data communicator 302 sends the monitoring data acquired by the data acquirer 301 to the data collection server 20 via a network. The data communicator 302 may be adapted to transmit the monitoring data in real time (i.e., each time the data acquirer 301 acquires the monitoring data), or at regular intervals (e.g., every 1 hour). The data communicator 302 may additionally or instead be adapted to transmit the monitoring data in response to a sending instruction from a user, at a specified time (e.g., 8 o'clock every morning), on a specified date (e.g., 1st day of every week or every month), and/or at a specified location (e.g., a medical facility during a visit).

Note that the user terminals 30 may each be any type of a device including a wearable type, a portable type, a stationary type, etc., as long as they are capable of acquiring the monitoring data from the corresponding patients. Also, each user terminal 30 may be a dedicated device exclusively for acquiring and transmitting the monitoring data, or a cellular phone (e.g., a smartphone), or a general-purpose computer (e.g., a tablet PC, a notebook PC, or a desktop PC). In the instances of adopting a cellular phone (such as a smartphone) or a general-purpose computer, processing behavior comparable with the dedicated device may be realized by installing and running a dedicated application program.

The data collection server 20 includes a processor 21. The processor 21 receives and processes the monitoring data of patients sent from the respective user terminals 30. Examples of the data processing which may be carried out here include encryption of at least personal information of each patient (e.g., patient's name) so that the contents of the monitoring data will not be comprehended by anyone but the medical staff conducting the remote monitoring for the patient concerned. While FIG. 1 assumes exemplary architecture where the data collection server 20 is an external cloud server or the like provided discretely from the medical information processing apparatus 10, the data collection server 20 may instead be arranged within the medical information processing apparatus 10 as a data collector. In the instances of having such a data collector in the medical information processing apparatus 10, the processor 21 may be omitted, and the monitoring data acquired from the user terminals 30 do not need to be processed.

Note that it is also possible for the medical information processing system 1 to omit data collection server 20 and adapt each user terminal 30 to directly send the monitoring data to the medical information processing apparatus 10.

The medical information processing apparatus 10 includes processing circuitry 11, a memory 12, an input interface 13, a communication interface 14, and a display 15. The processing circuitry 11, the memory 12, the input interface 13, the communication interface 14, and the display 15 are connected together via, for example, a bus so that they can communicate with each other.

The processing circuitry 11 is, for example, a processor functioning as a center of the medical information processing apparatus 10. The processing circuitry 11 is, for example, a processor such as a central processing unit (CPU), a graphics processing unit (GPU), etc. The processing circuitry 11 includes an acquisition function 111, a level calculation function 112, a level of need calculation function 113, a determination function 114, and a notification function 115.

The acquisition function 111, for example, decrypts each of the encrypted monitoring data of a plurality of patients, passed from the communication interface 14, by referring to the memory 12 so as to obtain usable monitoring data sets. When receiving non-encrypted monitoring data from the user terminals 30 or the data collection server 20, the acquisition function 111 may relay the monitoring data as it is for use in the later processing.

The level calculation function 112 calculates a personal condition level for the physical condition of each patient using the monitoring data.

The level of need calculation function 113 calculates levels of need for intervention based on the personal condition levels and a group condition level. The levels of need for intervention indicate how necessary a medical intervention (e.g., remote tutorial, face-to-face tutorial, prompting of a hospital visit, etc.) for the patient under remote monitoring is. The group condition level is a level representing a physical condition of the patient group including the patients concerned. Such a patient group is assumed to be a group of patients who are suffering from the same disease and cared for by the medical facility or the medical staff conducting the remote monitoring, but the patient group may also embrace patients suffering from the same disease inside the medical facility, or patients suffering from different diseases.

The determination function 114 determines or specifies a patient whose level of need for intervention satisfies a predetermined condition. The patient with an intervention degree satisfying a predetermined condition may also be called a “patient in need of intervention”, i.e., a patient for whom a medical intervention is needed.

The notification function 115 sends information for medical intervention to the user terminal 30 that belongs to the patient in need of intervention. The information for medical intervention here may be, for example, a message prompting a change in lifestyle or a visit to the hospital, information about a reservation for a diagnosis, and so on.

The memory 12 is a storage device adapted to store various sets of information, and examples of the memory 12 include a read-only memory (ROM), a random-access memory (RAM), a hard disk drive (HDD), a solid state drive (SSD), and an integrated circuit memory device. The memory 12 may also be adapted as a drive unit, etc. to read and write various information sets from and to portable storage media such as a CD-ROM drive, a DVD drive, and a flash memory. Note that it is not required to realize the memory 12 by a single storage device. For example, the memory 12 may be realized by multiple storage devices. The memory 12 may be located within an external computer connected to the medical information processing apparatus 10 via a network.

The memory 12 stores various items including a report preparation program according to the embodiment. In an exemplary implementation, this program may be prestored in the memory 12. In another exemplary implementation, the program may be distributed as an item stored in a non-transitory storage medium, and then read from the non-transitory storage medium to be installed in the memory 12.

The input interface 13 is adapted to receive various input operations from a user who is a member of the medical staff (e.g., a doctor) and convert the received input operations into electrical signals for output to the processing circuitry 11. The input interface 13 according to the embodiment is coupled to one or more input devices such as a mouse, a keyboard, a track ball, switches, buttons, a joystick, a touch pad, and a touch panel which allows input of commands through contact of an operation screen. Note that the input devices coupled to the input interface 13 may each be an input device arranged at an external computer connected via a network, etc.

The communication interface 14 receives encrypted monitoring data from the data collection server 20. The communication interface 14 also performs data communications with various entities including a hospital information system, an electronic health record system, a radiation section information system, a medical image management system (or a picture archiving and communication system (PACS)), etc. The communication interface 14 may be adapted to perform the data communications according to a preset known protocol. For example, communications complying with health level 7 (HL7) are performed between the medical information processing apparatus 10 and each of the hospital information system, the electronic health record system, and the radiation section information system. Also, communications complying with, for example, digital imaging and communications in medicine (DICOM) are performed between the medical information processing apparatus 10 and the medical image management system.

The display 15 displays various information items according to instructions from the processing circuitry 11. The display 15 may display graphical user interfaces (GUIs), etc., for accepting various operations from users. As the display 15, any display equipment may be discretionarily employed, including a cathode ray tube (CRT) display, a liquid crystal display, an organic EL display, an LED display, a plasma display, and a touch display which allows for touch input operations. However, the medical information processing apparatus 10 does not need to be furnished with the display 15, and the medical information processing apparatus 10 may instead cause an external display device to display intended GUIs, or present the GUIs via a projector or the like.

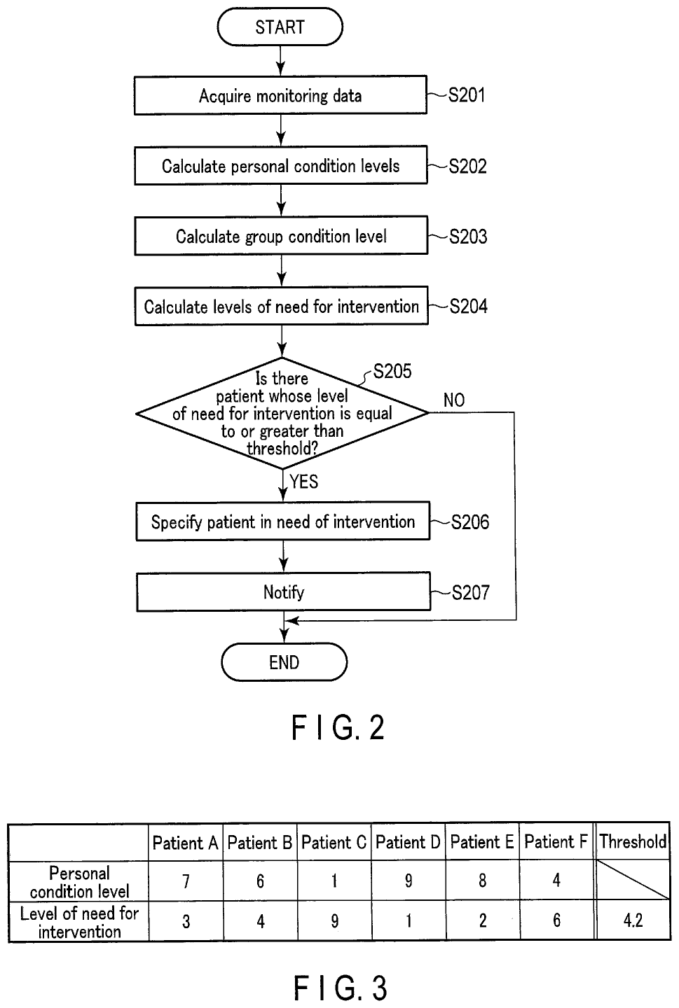

Next, an intervention determining process performed by the medical information processing apparatus 10 according to the first embodiment will be described with reference to the flowchart in FIG. 2.

The intervention determining process may be performed at specific intervals (e.g., once a day), or the timing to perform the intervention determining process may be set according to the type of disease associated with a patient group. For example, if heart failure is associated with a patient group, the intervention determining process for that group may be performed every day, and if high blood pressure is associated with a patient group, the intervention determining process may be performed once a week.

In step S201, the acquisition function 111 acquires monitoring data of each of the plurality of patients.

In step S202, the level calculation function 112 calculates a personal condition level for each patient based on the monitoring data. More specifically, the level calculation function 112 sets one or more parameters assumed to have an influence on the physical condition of a patient from the monitoring data, and calculates the personal condition level concerned. Such parameters may be, for example, vital signs and activity amounts contained in the monitoring data. Instead, or additionally, values obtained by quantifying the chief complaint declared by the patient himself/herself, or an objective finding of people close to the patient such as a care staff for the patient, etc. may be used as the parameters. Furthermore, values obtained by quantifying environmental information for the patient (e.g., ambient temperature and humidity), weather on the day of acquiring the monitoring data, information about the patient's living conditions (e.g., whether the patient is single or has a roommate), and so on may also be used as the parameters.

The level calculation function 112 calculates the personal condition level using one or more of the above discussed parameters by, for example, a deductive point system. As a concrete example, supposing that the personal condition level according to this calculation takes a value on a scale of 1 to 10, where a higher value indicates a better physical condition, the parameters may each be applied to a respective calculation formula that represents an abnormal condition. For example, if blood pressure data in the monitoring data is set as the parameter and shows a value equal to or higher than a predetermined value, “−1” is reflected in the level calculation.

In step S203, the level calculation function 112 calculates a group condition level. For example, the group condition level may be calculated for a group of patients who are under remote monitoring through the user terminals 30, or may be based on personal condition levels of the respective patients in a group associated with the same disease. The group condition level may be a concrete statistical value such as an average value, a median value, or the like of the multiple personal condition levels calculated in step S202. As another option, the group condition level may be calculated using a sole factor that is common to the grouped patients (e.g., weather, an event, the number of infected patients, etc.), or using the personal condition levels and the factor common to the grouped patients in combination. The event here is assumed to be a value obtained by quantifying, according to the empirical rules, the occurrence of diseases or the like that can be frequently seen on particular days or during particular periods (e.g., holidays, New Year's Day, winter, etc.). For example, increased events of cardiac disorder or brain disorder due to a shock from a sudden temperature change in a shower room, a lavatory, etc. are expected in winter, and accordingly, calculation of the group condition level may reflect such a period as one parameter.

In step S204, the level of need calculation function 113 calculates a level of need for intervention. The level of need for intervention may be, for example, a value obtained by subtracting a value of the personal condition level from a predetermined constant. As another option, the level of need for intervention may be calculated using any given mathematical function that includes the patient's personal condition level and the group condition level as variables. Such a mathematical function may provide, for example, a higher level of need for intervention for a personal condition level that is worse than the group condition level.

In step S205, the determination function 114 determines whether or not there is a patient whose level of need for intervention satisfies a predetermined condition. The determination function 114 here determines whether or not there is a patient whose level of need for intervention is equal to or higher than a threshold. If it is determined that there is a patient whose level of need for intervention is equal to or higher than the threshold, the process advances to step S206. If it is determined that there is no patient whose level of need for intervention is equal to or higher than the threshold, the process is terminated. Note that this threshold may be set based on levels of need for intervention as will be discussed, or set to a given value.

In step S206, the determination function 114 determines (specifies) a patient whose level of need for intervention is equal to or higher than the threshold as a patient in need of intervention.

In step S207, the notification function 115 informs the medical staff and/or the patient in need of intervention that an intervention should be done. When the medical staff is the notification target to be informed of the need for an intervention, the notification function 115 may, for example, cooperate with an applicable health record system and cause the display 15 to present information for the need for an intervention as, for example, a pop-up object. A setting is also possible where a window for the health record of the patient in need of intervention opens in response to the medical staff clicking this pop-up object. When the patient in need of intervention is the notification target to be informed of the need for an intervention, the notification function 115 may send information for a medical intervention to the corresponding user terminal 30 via the communication interface 14 of the medical information processing apparatus 10.

Note that FIG. 2 assumes an exemplary process where the determination for intervention is made every time the levels of need for intervention are calculated, but this is not a limitation. The determination for intervention may also use the results of previous and earlier calculations of the levels of need for intervention. For example, the determination function 114 may determine whether or not a subject level of need for intervention satisfies the predetermined condition using the average of the levels of need for intervention previously calculated and the levels of need for intervention calculated at present.

Also, the determination function 114 may specify a patient as a patient in need of intervention, if it has been consecutively determined multiple times that his/her level of need for intervention is equal to or higher than the threshold. In such a configuration, the determination function 114 in step S205 may also determine whether or not there is a patient whose level of need for intervention has been consecutively determined to be equal to or higher than the threshold a predetermined number of times from the previous or earlier determination step. Here, a predetermined number of consecutive determinations of the level of need for intervention being equal to or higher than the threshold is not necessarily required for specifying a patient in need of intervention. For example, it is also possible to specify a patient in need of intervention by determining whether or not the level of need for intervention has been determined to be equal to or higher than the threshold a predetermined number of times or more during a predetermined period.

As the predetermined condition, the determination function 114 may adopt, instead of the threshold, a degree of priority as to when to receive a medical intervention. In other words, the order of priority for the plurality of patients in need of intervention may be determined so that the patient with a higher level of need for intervention is assigned a higher priority, that is, this patient will be given a medical intervention prior to the others.

Next, FIG. 3 will be referred to for explaining a first exemplary setting of the levels of need for intervention and the threshold according to the first embodiment.

FIG. 3 is a table showing the personal condition level and the level of need for intervention for each of six patients A to F. Since patients with a high personal condition level are considered to be in a healthy condition, they do not need medical intervention and their levels of need for intervention are accordingly low. On the other hand, a patient with a low personal condition level is considered to be in a bad physical condition, and as such, he/she requires an intervention and has a high level of need for intervention. For the sake of explanation, the description will assume an instance where each level of need for intervention is a value obtained by subtracting a value of the personal condition level from a constant.

In the example shown in FIG. 3, a value obtained by subtracting a value of the personal condition level from “10” as a constant is set to the level of need for intervention. As a specific example, patient A has a personal condition level of “7”, and accordingly, his/her level of need for intervention is “3”. The respective levels of need for intervention for the other patients are calculated in the same manner.

The table in FIG. 3 shows the threshold, which is assumed to be a relative threshold determined according to the group condition level. The threshold in this example is assumed to be an average value of the levels of need for intervention of patients A to F, and is calculated to be “4.2”.

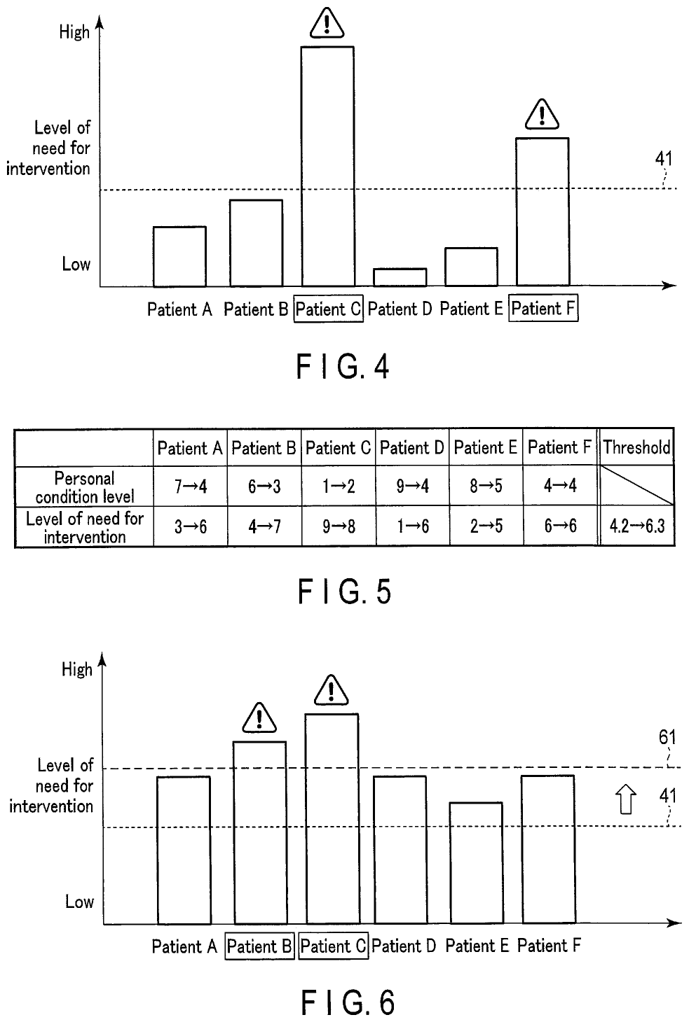

Next, FIG. 4 will be referred to for explaining a concept of the process for determining or specifying a patient in need of intervention, performed by the determination function 114 based on the first exemplary setting. FIG. 4 is a graph for the levels of need for intervention of the respective patients A to F and the threshold from FIG. 3. In this graph, the vertical axis represents the level of need for intervention, with the patients arranged along the horizontal axis. The broken line in the graph represents the threshold with a reference sign 41. Such a graphical representation is used for explaining the concept of the process for specifying a patient in need of intervention, and what is output by the determination function 114 does not need to be a graph. The determination function 114 may simply output information about the specified patient in need of intervention.

As shown in FIG. 4, patient C (level of need for intervention 9) and patient F (level of need for intervention 6) are patients whose level of need for intervention is equal to or higher than the threshold 41 (4.2), and accordingly, patient C and patient F are each specified as a patient in need of intervention, for whom a medical intervention should be done.

Next, reference will be made to FIG. 5, which is a table showing the levels of need for intervention and the threshold reflecting calculation in a process subsequent to the situation indicated by the table in FIG. 3.

For the sake of explanation, the values of the personal condition levels and the levels of need for intervention from FIG. 3 are also shown in FIG. 5 as the values calculated in the previous process. FIG. 5 assumes an instance where the patients have generally worsened their physical conditions and increased their levels of need for intervention. For example, the personal condition level of patient A has decreased from “7” to “4”, and accordingly, the level of need for intervention of patient A has been increased from “3” to “6”. Also, according to the increase of the levels of need for intervention, the threshold has also been increased from “4.2” to “6.3”.

FIG. 6 will be referred to, which shows a concept of the process for specifying a patient in need of intervention, performed by the determination function 114 based on the first exemplary setting shown in FIG. 5 for patients A to F.

Similarly to FIG. 4, what is shown in FIG. 6 is a graph showing the levels of need for intervention of the respective patients. As shown in FIG. 6, the threshold (6.3) that is denoted by reference sign 61 is now higher than the threshold 41 (4.2), and patient B (level of need for intervention of 7) and patient C (level of need for intervention of 8) are now patients whose level of need for intervention is equal to or higher than the threshold. If, for example, the threshold adopted in the situation shown in FIG. 6 were a fixed value equal to the threshold 41, all the patients would have been specified as patients in need of intervention. However, by calculating a relative threshold according to the group condition level as in the case with the threshold 61, it is possible to specify, with priority, the patients who require a medical intervention more than others. Therefore, with the configuration of varying the threshold according to the group condition level, the need to conduct diagnosis for many patients at once is reduced so that a rush of diagnosis activities can be prevented and the burden on the medical staff can be mitigated.

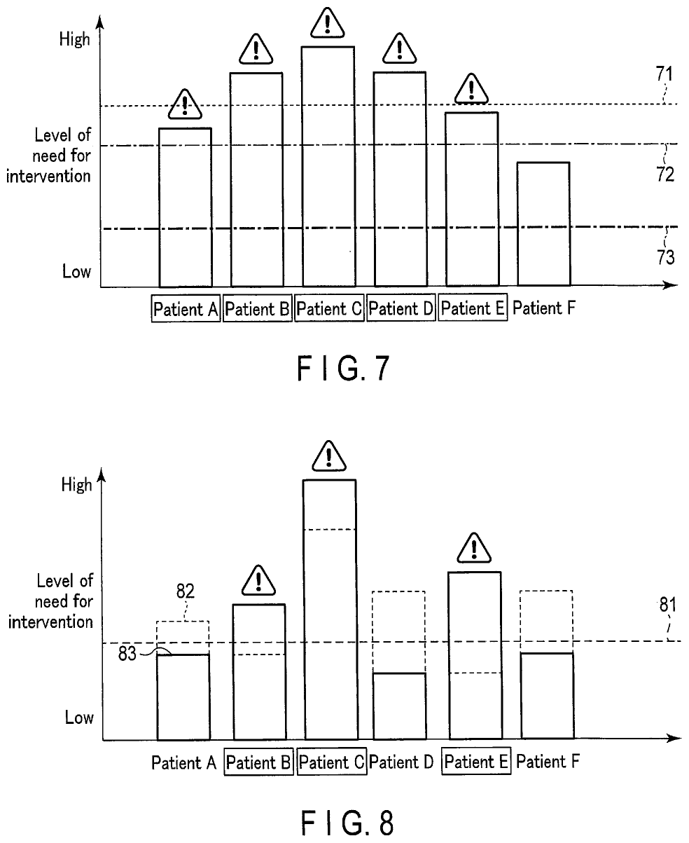

Next, FIG. 7 will be referred to for explaining a second exemplary setting of the levels of need for intervention and the threshold according to the first embodiment.

Similar to FIG. 4, what is shown in FIG. 7 is a graph showing the levels of need for intervention of the respective patients. FIG. 7 shows a threshold 71 which is a relative value that varies according to the group condition level and which may be the same as the threshold 41 shown in FIG. 4. In addition to the relative threshold 71, the second exemplary setting here includes absolute thresholds which are absolute values that do not vary according to the group condition level. As the absolute thresholds, two types are set, namely, an upper absolute threshold 72 and a lower absolute threshold 73.

The upper absolute threshold 72 is a threshold to indicate that an intervention is necessary irrespective of the value taken by the relative threshold 71. FIG. 7 assumes an instance where the relative threshold 71 takes a value higher than the upper absolute threshold 72. The determination function 114 here specifies patients B, C, and D, each having a level of need for intervention equal to or higher than the relative threshold 71, as patients in need of intervention. The determination function 114 further specifies that patients A and E having respective levels of need for intervention equal to or higher than the upper absolute threshold 72 are also patients in need of intervention, although their levels of need for intervention are not equal to or higher than the relative threshold 71.

Thus, with this configuration, even when the levels of need for intervention are generally high and such a situation would not allow patients to be easily specified as patients in need of intervention, setting an upper absolute threshold based on a common, established intervention judgment enables essential medical interventions to be conducted. Accordingly, patients' deteriorated physical conditions can be recognized and attended to in early stages.

Turning to the lower absolute threshold 73, this threshold is to indicate that an intervention is not necessary irrespective of the value taken by the relative threshold 71. For example, if the relative threshold 71 is set to a value lower than the lower absolute threshold 73, a patient is not specified as a patient in need of intervention unless his/her level of need for intervention is equal to or higher than both the relative threshold 71 and the lower absolute threshold 73. That is, when the level of need for intervention does not exceed the lower absolute threshold 73, it can be determined that a medical intervention is not necessary, even if the relative threshold 71 is exceeded.

When the levels of need for intervention of a patient group is generally low, that is, when the grouped patients are generally in good physical condition, the relative threshold 71 will likely take a low value. In such instances, setting the lower absolute threshold 73 can avoid medical interventions being conducted for patients who would normally not require any medical interventions according to an established intervention judgment. Accordingly, unnecessary burden on the medical staff can be eliminated or reduced.

Next, FIG. 8 will be referred to for explaining a third exemplary setting of the levels of need for intervention and the threshold according to the first embodiment.

Similarly to FIG. 4, what is shown in FIG. 8 is a graph showing the levels of need for intervention of the respective patients. The threshold in the third exemplary setting is a fixed threshold 81, and the level of need calculation function 113 calculates the levels of need for intervention using a mathematical function that varies according to the group condition level. For example, if, in a patient group, patients who are improving their physical conditions account for a large proportion of patients in the group, a mathematical function that increases the level of need for intervention of a patient having a deteriorating or constant physical condition is used.

In FIG. 8, broken lines 82 indicate the levels of need for intervention calculated in the previous intervention determining process, and solid lines 83 indicate the levels of need for intervention calculated in the current intervention determining process. Also, it will be assumed that the physical conditions of patients A, D, and F are recovering when the current intervention determining process is performed, while the other patients show no improvement in physical condition.

In the previous process, patients A, C, D, and F, each having a level of need for intervention equal to or higher than the threshold 81, were specified as patients in need of intervention. In the current process, since the physical conditions of patients A, D, and F have been recovering, and the group condition level has improved accordingly, the levels of need for intervention of the respective patients B, C and E without a physical condition improvement increase relatively. Consequently, even if patients B and E have not experienced a substantial change in physical condition, a relative judgment within the patient group is made for the need for a medical intervention, and patients B, C and E are specified as patients in need of intervention in the current process.

Note that the level of need calculation function 113 may perform calculation of the levels of need for intervention using a weighting according to, for example, a distance from the patient's residence to the applicable medical facility. As one example, it can be considered that a patient whose home is far from the medical facility might have difficulty in frequently seeing their doctor, depending on their physical condition. The level of need calculation function 113 in this case may calculate the level of need for intervention with a weighting so that a patient at a residence that is more distant from the medical facility is given a lower level of need for intervention.

In contrast, since it is not easy for patients whose home is far from the medical facility to repeatedly visit the medical facility for a diagnosis, there may be instances where, depending on the patient's physical condition, an earliest-possible medical intervention is preferred to prevent the physical condition from becoming severe. The level of need calculation function 113 here may calculate the level of need for intervention with a weighting so that a patient at a residence that is more distant from the medical facility is given a higher level of need for intervention.

As another example, the level of need calculation function 113 may perform calculation of the levels of need for intervention using a weighting according to a reservation status in the schedule of the medical facility. For example, there may be instances where a day or a period which is expected to be reserved for diagnosis by a patient specified as a patient in need of intervention already has a given number of reservations or more set (e.g., when many others reserve the day or the period for their diagnosis, or when many surgical operations are scheduled on the day or during the period). In such instances, if the patient in need of intervention is accepted for diagnosis on the same day or during the same period, a burden on the medical staff would increase. Accordingly, the level of need calculation function 113 may calculate the level of need for intervention with a weighting so that the calculated level of need for intervention has a lower value when there are many reservations in the schedule of the medical facility.

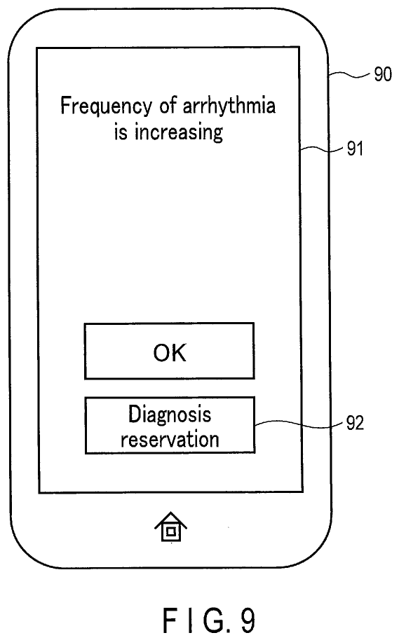

Next, FIG. 9 will be referred to for explaining one example of notification to the user terminal 30, performed by the notification function 115.

The example shown in FIG. 9 assumes that a notification from the medical information processing apparatus 10 to a patient in need of intervention is presented through a display screen of a smartphone 90 as the corresponding user terminal 30. In this example, a message 91 (“Frequency of arrhythmia is increasing.”) is presented together with a confirmation button (“OK”) and a diagnosis reservation button 92 (for transitioning to a diagnosis reservation screen). A setting may be done in advance so that, upon confirming such a message 91 and touching the diagnosis reservation button 92, the patient is guided to the diagnosis reservation screen for the medical facility. Accordingly, the patient can smoothly make a diagnosis reservation, and a corresponding medical intervention can be conducted at an appropriate timing.

In addition to a message as shown in FIG. 9, which is presented for recognition of the patient's condition, the notification function 115 may also present, for example, a message for expressly indicating that the message recipient is a patient in need of intervention, a message prompting a visit to the hospital, and so on. Moreover, messages such as instructions about the number and amount of doses, suggestions to increase the frequency of measurement and reporting of the monitoring data, etc., may also be presented. Accordingly, suitably tailored medical interventions that include not only diagnosis activities through a hospital visit or a home visit but also a follow-up assessment based on remote monitoring can be realized.

According to the first embodiment described above, levels of need for intervention of respective patients are calculated and a patient in need of intervention is specified based on personal condition levels and a group condition level. Accordingly, when the group condition level is good, it is possible to perform a control so that a patient showing a personal condition level that is not low in absolute value but is low in relative value, namely, a patient who will potentially experience a deteriorated physical condition, can be specified as a diagnosis subject. On the other hand, when the group condition level is bad, it is possible to specify, with priority, a patient who should be subjected to a medical intervention ahead of others. Therefore, the need to conduct diagnosis for many patients at once is reduced so that a concentration of the diagnosis activities can be prevented and the burden on the medical staff can be mitigated.

That is, the embodiment can save medical staff from irregular troublesome events while maintaining improved prognosis of patients, and consequently, the embodiment can assist implementation of effectively planned medical interventions. Moreover, with the configuration of sending a message that the message recipient is a patient in need of intervention or a message prompting a diagnosis reservation, etc. to the user terminal owned by the patient concerned, the embodiment enables medical interventions to be conducted at appropriate timings.

### Second Embodiment

The second embodiment differs from the first embodiment in that it has a configuration of setting, when a diagnosis is not available for patients in need of intervention, supplemental information for these patients. For example, if many patients are specified as patients in need of intervention and their diagnoses are expected to be on the day for which many reservations for diagnosis in the medical facility have already been made, or on which an emergency case breaks in, there is a possibility that not all the patients in need of intervention will receive the diagnosis. Accordingly, the supplemental information is set so that a patient in need of intervention who cannot receive a diagnosis will be able to receive a diagnosis with priority on the next day or later. Thereby, implementation of appropriate medical interventions can be continued.

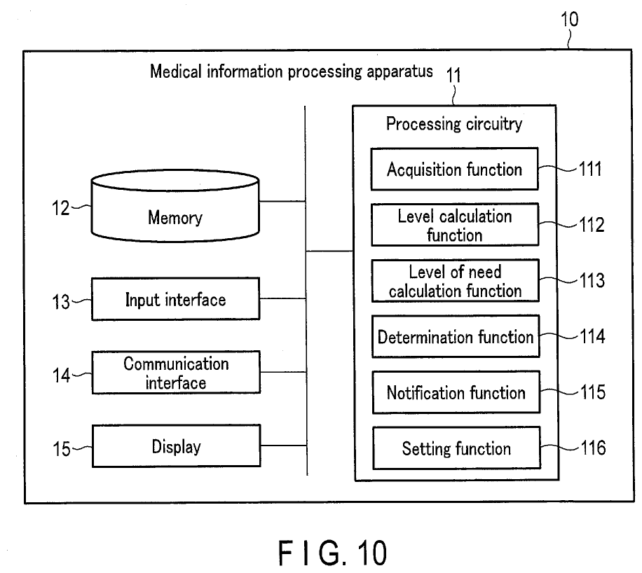

FIG. 10 is a block diagram showing a medical information processing apparatus 10 according to the second embodiment.

The medical information processing apparatus 10 according to the second embodiment includes processing circuitry 11, a memory 12, an input interface 13, a communication interface 14, and a display 15. The processing circuitry 11 includes an acquisition function 111, a level calculation function 112, a level of need calculation function 113, a determination function 114, a notification function 115, and a setting function 116.

The configurations other than the setting function 116 are the same as those of the medical information processing apparatus 10 according to the first embodiment, so their description will be basically omitted.

The setting function 116 extracts patients for whom a medical intervention is not available, from patients in need of intervention whose levels of need for intervention have satisfied a predetermined condition. This patient extracting process may be, for example, a process of extracting a patient whose electronic health record in the electronic health record system shows no medical interventions or diagnoses, etc., since the day on which he or she was specified as a patient in need of intervention. For each of the extracted patients, the setting function 116 sets supplemental information indicating that no medical intervention has been conducted for the patient in need of intervention. The setting function 116 may discard the supplemental information once the medical intervention for the patient for whom the supplemental information was set is completed.

As the supplemental information, information indicating that a patient was specified in the previous process as a patient in need of intervention but has not yet received a medical intervention may be presented through the display 15 of the medical information processing apparatus 10, using a mark (e.g., a flag) or a text message. The supplemental information may also indicate that a medical intervention for this patient should be performed with priority next. The supplemental information may be presented in cooperation with the electronic health record system in such a manner that the information indicating that a patient was specified in the previous process as a patient in need of intervention is displayed as a mark and/or a text message in association with the electronic health record of the patient.

When the supplemental information is sent to the user terminal 30 with a notification, the supplemental information may be presented through the screen of the user terminal 30 as a mark and/or a text message so that, for example, the patient can comprehend that the patient's level of need for intervention of the previous day was equal to or higher than the threshold, aside from the level of need for intervention of the day of the notification.

Note that, when the level of need calculation function 113 calculates again the level of need for intervention of a patient for whom the supplemental information has been set, the level of need calculation function 113 may use a weighting so that the calculated level of need for intervention will be higher.

According to the second embodiment described above, supplemental information is set in response to determining that no medical intervention has been done for a patient who was specified as a patient in need of intervention. Thus, supposing that a patient was specified as a patient in need of intervention but unable to receive a diagnosis on the first day, even if the patient then improved his/her physical condition and was not specified as a patient in need of intervention in the intervention determining process on the second day, it is possible to comprehend from the supplemental information that the patient was specified as a patient in need of intervention. Accordingly, the medical facility is enabled to conduct proper medical interventions for the patients for whom supplemental information has been set, by subjecting them to a diagnosis ahead of others, or performing other prioritized support.

The functions 111 to 116 according to the foregoing embodiments are not limited to implementations through a single processing circuitry component. Multiple independent processors may be employed together to form the processing circuitry so that the processors run programs to realize the functions 111 to 116. Also, the functions 111 to 116 may each be stored as a program in the memory 12, etc., so that the processing circuitry 11 executes the programs to realize the functions corresponding to the respective programs.

Moreover, the functions according to the embodiments may also be realized by installing programs for executing the processes described in connection with the embodiments in a computer such as a work station, and loading the programs on a space in the memory. Here, the programs for causing the computer to execute the processes may be stored in a storage medium, such as a magnetic disk (a hard disk, etc.), an optical disk (a CD-ROM, a DVD, Blu-ray Disc™), and a semiconductor memory, and distributed.

According to at least one embodiment described above, it is possible to save medical staff from irregular occurrences of burden-imposing events, while maintaining improved prognosis of patients.

While certain embodiments have been described, they have been presented by way of example only, and are not intended to limit the scope of the inventions. Indeed, the novel embodiments described herein may be embodied in a variety of other forms. Furthermore, various omissions, substitutions, and changes in the form of the embodiments may be made without departing from the spirit of the inventions. The accompanying claims and their equivalents are intended to cover such forms or modifications as would fall within the scope and spirit of the inventions.

1. A medical information processing apparatus comprising processing circuitry configured to:
acquire monitoring data of a patient;
calculate a personal condition level for a physical condition of the patient using the monitoring data;
calculate a level of need for intervention indicative of a degree of need for a medical intervention for the patient, based on the personal condition level and a group condition level for a patient group including the patient; and
determine whether or not the level of need for intervention of the patient satisfies a predetermined condition.

2. The medical information processing apparatus according to claim 1, wherein the predetermined condition is set according to the group condition level.

3. The medical information processing apparatus according to claim 1, wherein the processing circuitry calculates the level of need for intervention according to the group condition level.

4. The medical information processing apparatus according to claim 1, wherein
the predetermined condition indicates a first threshold, and
the processing circuitry determines whether or not the level of need for intervention of the patient is equal to or higher than the first threshold.

5. The medical information processing apparatus according to claim 1, wherein
the predetermined condition indicates a first threshold as a relative value set according to the group condition level and a second threshold as an absolute value not varying according to the group condition level, the second threshold being a reference for determining that the medical intervention is required, and
the processing circuitry determines whether or not the level of need for intervention of the patient is equal to or higher than the first threshold or the second threshold.

6. The medical information processing apparatus according to claim 1, wherein
the predetermined condition indicates a first threshold as a relative value set according to the group condition level and a third threshold as an absolute value not varying according to the group condition level, the third threshold being a reference for determining that the medical intervention is not required, and
the processing circuitry determines whether or not the level of need for intervention of the patient is equal to or higher than the first threshold and the third threshold.

7. The medical information processing apparatus according to claim 1, wherein
the predetermined condition comprises a priority as to when to receive the medical intervention, and
the processing circuitry determines that the higher the level of need for intervention, the higher the priority.

8. The medical information processing apparatus according to claim 1, wherein the processing circuitry calculates the level of need for intervention using a weighting according to a distance from a residence of the patient to a medical facility.

9. The medical information processing apparatus according to claim 1, wherein the processing circuitry calculates the level of need for intervention using a weighting according to a reservation status in a schedule of a medical facility.

10. The medical information processing apparatus according to claim 1, wherein the processing circuitry is further configured to set supplemental information when the medical intervention has not been performed for a patient in need of intervention as the patient whose level of need for intervention satisfies the predetermined condition, the supplemental information indicating that the medical intervention has not been performed for the patient in need of intervention.

11. The medical information processing apparatus according to claim 1, wherein the processing circuitry is further configured to send information for the medical intervention to a patient in need of intervention as the patient whose level of need for intervention satisfies the predetermined condition.

12. The medical information processing apparatus according to claim 11, wherein the processing circuitry sends information about a diagnosis reservation to the patient in need of intervention.

13. A medical information processing method comprising:
acquiring monitoring data of a patient;
calculating a personal condition level for a physical condition of the patient using the monitoring data;
calculating a level of need for intervention indicative of a degree of need for a medical intervention for the patient, based on the personal condition level and a group condition level for a patient group including the patient; and
determining whether or not the level of need for intervention of the patient satisfies a predetermined condition.

14. A medical information processing system comprising a plurality of user terminals, a data collection server, and a medical information processing apparatus, wherein
the user terminals are each configured to
acquire monitoring data of a patient, and
send the monitoring data to the data collection server,
the data collection server is configured to encrypt the monitoring data sent from the user terminals, and
the medical information processing apparatus comprises processing circuitry configured to
receive the encrypted monitoring data from the data collection server,
decrypt the encrypted monitoring data to obtain the monitoring data,
calculate a personal condition level for a physical condition of the patient using the monitoring data,
calculate a level of need for intervention indicative of a degree of need for a medical intervention for the patient, based on the personal condition level and a group condition level for a patient group including the patient, and
determine whether or not the level of need for intervention of the patient satisfies a predetermined condition.
